(19) United States
(12) Patent Application Publication
(10) Pub. No.: US 2024/0245452 A1
(43) Pub. Date: Jul. 25, 2024

(54) SINGLE PASS LARGE BORE TRANSSEPTAL CROSSING

(71) Applicant: CIRCA SCIENTIFIC, INC., ENGLEWOOD, CO (US)

(72) Inventors: Brian Schuler, York, PA (US); Steven Howard, La Jolla, CA (US); Keith Weier, San Marcos, CA (US); Matthew Spencer Toone, Sandy, UT (US)

(73) Assignee: CIRCA SCIENTIFIC, INC., ENGLEWOOD, CO (US)

(21) Appl. No.: 18/418,119

(22) Filed: Jan. 19, 2024

Related U.S. Application Data
(60) Provisional application No. 63/480,618, filed on Jan. 19, 2023.

(51) Int. Cl. A61B 18/14; A61B 18/00

(52) U.S. Cl. CPC A61B 18/1492; A61B 2018/00077; A61B 2018/00083; A61B 2018/00351

(57) ABSTRACT

A transseptal crossing system can have a sheath and an elongate tubular body. The tubular body can include an electrically conductive sidewall defining a central lumen and a distal end section. The central lumen may be configured to receive a guidewire and to permit at least a portion of the guidewire to extend through a distal opening of the electrically conductive sidewall. The distal end section can comprise a distal end surface in electrical communication with the electrically conductive sidewall. The distal end surface includes a first section being positioned generally perpendicular to a longitudinal axis of the elongate tubular body and a second section being positioned at a non-orthogonal angle relative to the longitudinal axis of the elongate tubular body. At least one of the first section or the second section may be configured to deliver energy to a target tissue.

### CROSS-REFERENCE TO RELATED APPLICATIONS

This application claims priority to U.S. Provisional Patent Application No. 63/480,618, filed on Jan. 19, 2023, under 35 U.S.C. § 119(e).

This application incorporates by reference the entirety of each of the following applications herein in their entirety forming party of the present disclosure: U.S. patent application Ser. Nos. 16/896,582; 16/896,604; and 16/896,648, filed on Jun. 9, 2020, which each claim the priority benefit under 35 U.S.C. § 119(e) of U.S. Provisional Patent Application No. 63/024,986, filed on May 14, 2020. Any feature, structure, material, method, or step that is described and/or illustrated in any embodiment in any of the foregoing patent applications can be used with or instead of any feature, structure, material, method, or step that is described and/or illustrated in the following paragraphs of this specification or the accompanying drawings.

### INTRODUCTION

Transseptal crossing is used to access the left atrium crossing from the right atrium through the septal wall for any of a variety of EP or structural heart procedures. For example, the left atrium is routinely accessed to assess hemodynamics and/or perform mitral valvuloplasty, or to accommodate transvascular atrial fibrillation (AF) ablation procedures.

Crossing the septum normally requires locating and puncturing the fossa ovalis to access the left atrium. Locating the fossa ovalis may be accomplished using fluoroscopy and ultrasound, and potentially echocardiography.

Mechanical puncture through the tissue of the fossa ovalis can be accomplished using a piercing tool such as a standard Brockenbrough needle as is understood in the art. Alternatively, a transseptal needle having a radio frequency energized tip may be used, such as those produced by Baylis Medical Company, Inc.

The foregoing devices and techniques have proven useful in a variety of EP and structural heart procedures, in which the procedural access sheath is often no larger than about 11 French. However, a growing number of procedures such as left atrial appendage occlusion device implantation and various Mitral valve replacement or repair require “large bore” access, which is not possible using the above techniques alone.

Instead, traditional transseptal puncture is normally carried out with small bore sheaths ranging from 8 Fr to 11 Fr which are then exchanged over stiff guidewires for the large bore sheaths with outer dimensions as large as 24 Fr in the case of the MitraClip Steerable Guide Catheter. Dilators for the transseptal sheaths usually accommodate 0.032 wires, however Baylis has introduced a small bore transseptal sheath with a dilator that accommodates a 0.035 wire. Regardless of the transseptal sheath, the operator must utilize a number of additional pieces of equipment including the small bore transseptal sheath and dilator, and often a 0.025 “stiff” pigtail wire (Baylis, Toray) to use as a rail to drive the small bore transseptal sheath and dilator before switching to a 0.035 guidewire (Amplatz extra stiff or Safari) to drive the large bore sheath safely into the LA. The entire small bore sheath must be driven into the LA to make the switch to the appropriate stiff 0.035 guidewire to drive the large bore sheath.

Thus, there remains a need for a transseptal crossing system that is based upon a 0.035 inch guidewire access, that enables single pass crossing of larger sheaths, such as the Boston Scientific Watchman guide sheath, Medtronic Flexcath and others known in the art or yet to be released.

### SUMMARY

This Summary is provided to introduce a selection of concepts in a simplified form that are further described below in the Detailed Description. This Summary is not intended to identify key features or essential features, nor is it intended to limit the scope of the claims included herewith.

In general, the disclosure provides a single pass transseptal crossing device, for enabling large bore access in a single pass. The system includes an insulated cannula with a conductive tip positionable through the dilator of a large bore sheath. The tip of the cannula is expressed distal to the tip of the dilator. The insulated cannula can serve as an electrical conduit of RF energy. The cannula can be energized to deliver RF energy directly to tissues and/or through a separate conductive wire or obturator. An insulated wire or obturator can thus be independently energized or can be energized passively via the energized cannula. The cannula may be 0.050″ OD and approximately 0.038″ ID allowing delivery of a 0.035″ wire which together (stiff 0.035 wire and 0.050 cannula) creates a sturdy rail to drive large bore dilators and sheaths in a single pass. The tip of the cannula can be non-orthogonal to deliver high density current to an angled edge of the cannula for improved cutting. The system permits irrigation with hypotonic saline or D5W to preferentially drive current through the myocardium.

Thus, there is provided in accordance with an embodiment of a single pass, large bore transseptal crossing catheter, such as for accessing the left atrium of the heart. The catheter comprises an elongate, flexible tubular body, having a proximal end, a distal end and an electrically conductive sidewall defining a central lumen. An insulation layer surrounds the sidewall and leaving exposed a first distal electrode tip. An inner conductive wire having a second distal electrode tip, is axially movably extendable through the central lumen. A tubular insulation layer is provided in between the wire and the electrically conductive sidewall.

The first distal electrode tip may comprise an annular conductive surface at the distal end of the tubular body. The second distal electrode tip may be concentrically extendable through the annular conductive surface. The first distal electrode tip may comprise at least one distal projection, or at least two or three projections and in one implementation comprises a non-orthogonal distal edge.

The second distal electrode may comprise a smooth, hemispherical surface, or may be provided with a sharpened distally facing projection. The catheter may additionally comprise an annular lumen extending between the tubular body and the wire, from the proximal hub to an exit port at the distal end.

In accordance with a further embodiment, there is provided an introducer sheath, for enabling a single pass, large bore transseptal crossing. The introducer sheath comprises an elongate, flexible tubular body, having a proximal end, a distal end and an electrically conductive sidewall defining a central lumen. A tubular insulation layer may surround the sidewall, leaving exposed an annular conductive surface at the distal end. A proximal hub may be provided on the tubular body, having at least one access port in communication with the central lumen. A connector may be carried by the proximal end, in electrical communication with the conductive sidewall. The distal conductive surface may comprise at least one distal projection, or at least two or three projections and in one implementation comprises a non-orthogonal distal edge.

There is also provided a method of accessing the left atrium with a large bore catheter in a single pass. The method comprises providing a single pass, large bore transseptal crossing catheter; positioning the distal end in contact with a fossa ovalis; and energizing the distal end to enable passage of the distal end into the left atrium.

The energizing step may comprise energizing a first distal electrode tip on a conductive tubular cannula and/or energizing a second distal electrode tip on an RF core wire. The first and second distal electrode tips may be energized in a bipolar mode.

The method may comprise accessing the left atrium, energizing the second distal electrode tip, advancing the wire through the fossa ovalis, thereafter energizing the first distal electrode tip and advancing the cannula into the left atrium.

A large bore access sheath may thereafter be advanced over the transseptal crossing catheter and into the left atrium, and the transseptal crossing catheter may then be removed, leaving the large bore access sheath extending into the left atrium.

An index procedure catheter may be advanced through the large bore access sheath and into the left atrium. The index procedure catheter may be configured to deliver a left atrial appendage implant such as an occlusion device, or to accomplish a mitral valve repair or replacement.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 schematically illustrates a transseptal crossing system.

FIG. 2 is a side elevational view of an RF needle.

FIG. 2A is a cross section taken along the line A-A in FIG. 2.

FIG. 2B is a detail view of the distal tip of the needle in FIG. 2.

FIG. 3 is a cross sectional view through the needle of FIG. 2.

FIG. 4A-4C are detail views of the distal energy delivery tip of one implementation.

FIG. 5A-5C are detail views of the distal energy delivery tip of another implementation.

FIG. 6 is a schematic cross section of a portion of a human heart, having a transseptal crossing system positioned in the right atrium.

FIG. 7 is a view as in FIG. 6, showing positioning of the distal tip of the transseptal crossing system at the fossa ovalis.

FIG. 8 shows penetration of the guidewire and cannula through the fossa ovalis.

FIG. 9 shows penetration of the large bore sheath and dilator crossing the fossa ovalis.

FIG. 10 shows the large bore sheath in position across the septum with the dilator and other system components removed to provide access to the left atrium.

FIG. 11 illustrates an embodiment of a distal energy delivery tip.

FIG. 12 illustrates an embodiment of a distal energy delivery tip.

FIG. 13 illustrates an embodiment of a distal energy delivery tip.

FIG. 14 illustrates an embodiment of a distal energy delivery tip.

FIG. 15 illustrates an embodiment of a distal energy delivery tip.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

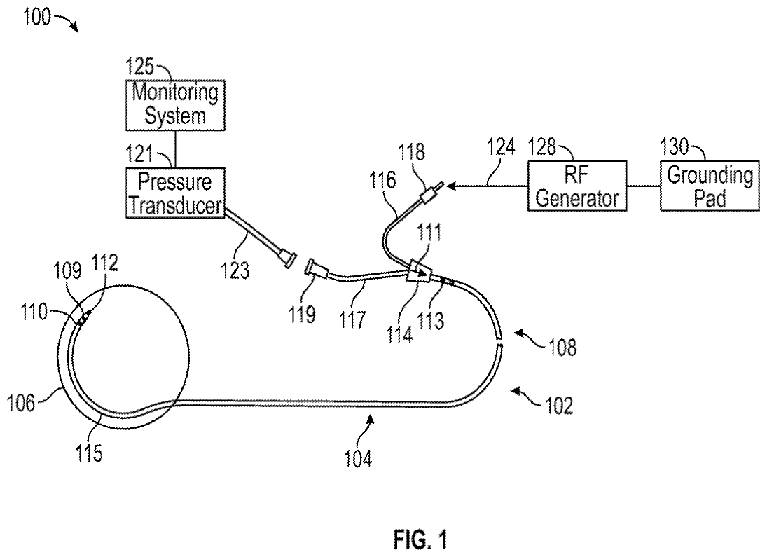

FIG. 1 illustrates an embodiment of a tissue penetrating apparatus 102 in a transseptal crossing system 100. Apparatus 102 comprises an elongate tubular body 104 having a distal region 106, and a proximal region 108. Distal region 106 is adapted to be inserted within and along a lumen of a body of a patient, such as a patient's vasculature, and maneuverable therethrough to a desired location proximate material, such as tissue, to be perforated.

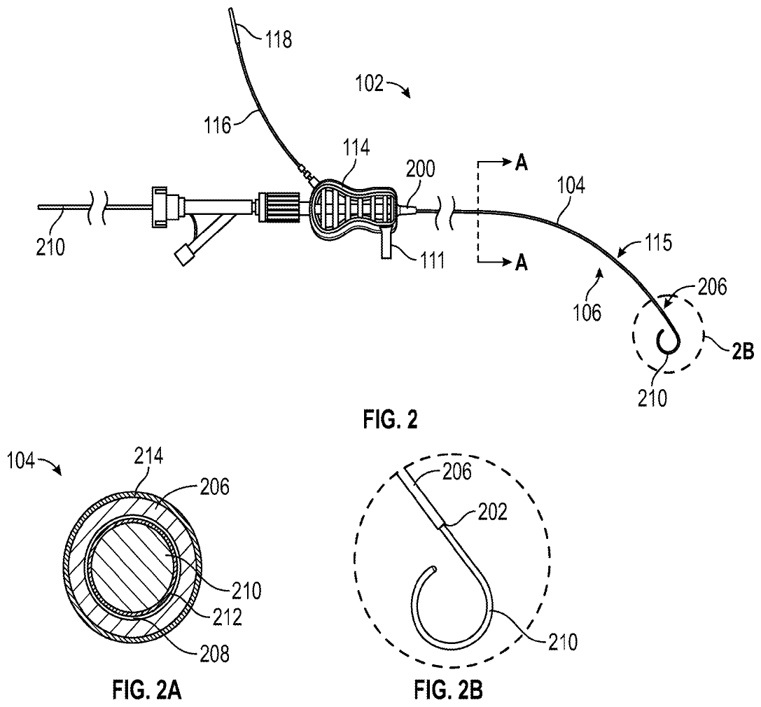

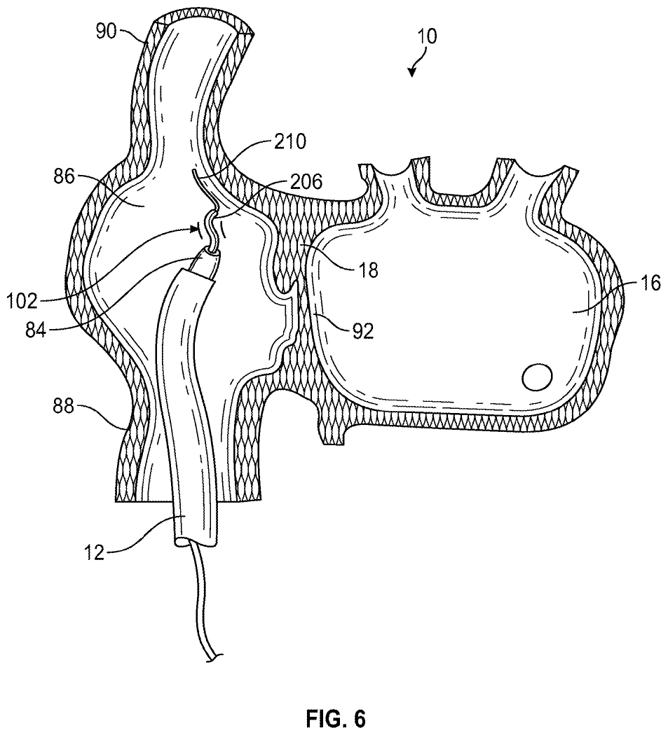

In some embodiments, the tubular body 104 may have at least one lumen extending from proximal region 108 to distal region 106 such as lumen 208 shown in FIG. 2A. Tubular body 104 may be constructed of a biocompatible polymer material jacket typically with a metal core that provides column strength to apparatus 102. The tubular body 104 may be sufficiently stiff to permit a dilator 84 and a large bore guiding sheath 12 (See FIG. 6) to be easily advanced over apparatus 102 and through a perforation. Examples of suitable materials for the tubular portion of tubular body 104 are stainless steel, nitinol, polyetheretherketone (PEEK), nylon, and polyimide. In the illustrated embodiment, the outer diameter along the tubular portion of tubular body 104 may taper down to distal region 106. In alternate embodiments, the outer diameter along tubular body 104 remains substantially constant from proximal region 108 to distal region 106.

Distal region 106 comprises a softer polymer material with an optional embedded braid or coil so that it is pliable and atraumatic when advanced through vasculature. In some embodiments, the material is also formable (e.g., Nitinol or stainless steel with a polymer jacket), so that its shape can be changed during manufacturing, typically by exposing it to heat while it is fixed in a desired shape. In an alternate embodiment, the shape of distal region is modifiable by the operator during use. An example of a suitable plastic is PEBAX (a registered trademark of Atofina Chemicals, Inc.). In the present embodiment, the distal region 106 comprises a curve portion 115.

As the distal region 106 is advanced out of a guiding sheath, it may have a preset curve so that it curls away from the general axis of the sheath which helps ensure that energy delivery tip 112 is not in a position to inadvertently injure unwanted areas within a patient's heart after trans-septal perforation. Curve length may be about 4 cm (about 1.57″) to about 6 cm (about 2.36″) and the curve may traverse about 225 to about 315 degrees of the circumference of a circle. For example, the curve may be about 5 cm in length and may traverse about 270 degrees of the circumference of a circle. Such an embodiment may be useful to avoid unwanted damage to cardiac structures.

In some embodiments, curve portion 115 begins about 0.5 cm to about 1.5 cm proximal to energy delivery tip 112, leaving an approximately 1 cm (about 0.39″) straight portion in the distal region 106 of apparatus 102. This ensures that this initial portion of apparatus 102 will exit dilator 84 (see FIG. 6) without curving, enabling the operator to easily position the apparatus 102, for example, against a septum as described further below. This feature further ensures that the distal region 106 of apparatus 102 will not begin curving within the atrial septum.

Distal region 106 may have a smaller outer diameter compared to the remainder of tubular body 104 so that dilation of a perforation is limited while the distal region 106 is advanced through the perforation. Limiting dilation seeks to ensure that the perforation will not cause hemodynamic instability once apparatus 102 is removed. In some embodiments, the outer diameter of distal region 106 may be no larger than about 0.8 mm to about 1.0 mm. For example, the outer diameter of distal region 106 may be about 0.9 mm (about 0.035″). This is comparable to the distal outer diameter of the trans-septal needle that is traditionally used for creating a perforation in the atrial septum. Similarly, in some embodiments, the outer diameter of tubular body 104 may be no larger than about 0.040″ to about 0.060″. For example, the outer diameter of tubular body 104 may be about 0.050″ (1.282 mm), which is also comparable to the trans-septal needle dimensions.

Distal region 106 terminates at functional tip region 110, which comprises an energy delivery component and optionally also as an ECG measuring device. Functional tip region 110 comprises at least one energy delivery tip 112 made of a conductive and optionally radiopaque material, such as stainless steel, tungsten, platinum, or another metal. One or more radiopaque markings may be affixed to tubular body 104 to highlight the location of the transition from distal region 106 to the remainder of tubular body 104, or other important landmarks on apparatus 102. Alternately, the entire distal region 106 of apparatus 102 may be radiopaque. This can be achieved by filling the polymer material, for example PEBAX, used to construct distal region 106 with radiopaque filler. An example of suitable radiopaque filler is Bismuth. Distal region 106 may contain at least one opening, for example, exit port 109, which is in fluid communication with main lumen 200 (FIG. 2A) as described further below.

In embodiment illustrated in FIG. 1, proximal region 108 comprises a hub 114, to which are attached a catheter connector cable 116, and connector 118. Tubing 117 and adapter 119 may be attached to hub 114 as well. Proximal region 108 may also have one or more depth markings 113 to indicate distances from functional tip region 110, or other important landmarks on apparatus 102. Hub 114 comprises a curve direction or orientation indicator 111 that is located on the same side of apparatus 102 as the curve portion 115 in order to indicate the direction of curve portion 115. Orientation indicator 111 may comprise inks, etching, or other materials that enhance visualization or tactile sensation. One or more curve direction indicators may be used and they may be of any suitable shape and size and a location thereof may be varied about the proximal region 108.

As shown in FIG. 1, adapter 119 is configured to releasably couple apparatus 102 to an external pressure transducer 121 via external tubing 123. External pressure transducer 121 is coupled to a monitoring system 125 that converts a pressure signal from external pressure transducer 121 and displays pressure as a function of time. Catheter connector cable 116 may connect to an optional Electro-cardiogram (ECG) interface unit via connector 118. An optional ECG connector cable connects an ECG interface unit to an ECG recorder, which displays and captures ECG signals as a function of time. A generator connector cable may connect the ECG interface unit to an energy source such as a generator (not illustrated). In this embodiment, the ECG interface unit can function as a splitter, permitting connection of the electrosurgical tissue piercing apparatus 102 to both an ECG recorder and generator simultaneously. ECG signals can be continuously monitored and recorded and the filtering circuit within the ECG interface unit and may permit energy, for example RF energy, to be delivered from generator 128 through electrosurgical apparatus 102 without compromising the ECG recorder.

In another embodiment (not shown) of apparatus 102, there may be a deflection control mechanism associated with the distal region 106 of apparatus 102 and an operating mechanism to operate said control mechanism associated with the proximal region 108 of apparatus 102. One or two or more pull wires may extend from a proximal control to the distal region 106 to actively deflect the distal region 106 as will be understood in the art. The control mechanism may be used to steer or otherwise actuate at least a portion of distal region 106.

Generator 128 may be a radiofrequency (RF) electrical generator that is designed to work in a high impedance range. Because of the small size of energy delivery tip 112 the impedance encountered during RF energy application is very high. General electrosurgical generators are typically not designed to deliver energy in these impedance ranges, so only certain RF generators can be used with this device. In one embodiment, the energy is delivered as a continuous wave at a frequency between about 400 kHz and about 550 kHz, such as about 460 kHz, a voltage of between 100 to 200 V RMS and a duration of up to 99 seconds. A grounding pad 130 is coupled to generator 128 for attaching to a patient to provide a return path for the RF energy when generator 128 is operated in a monopolar mode.

Other embodiments could use pulsed or non-continuous RF energy. Some embodiments for pulsed radio frequency energy have radio frequency energy of not more than about 60 watts, a voltage from about 200 Vrms to about 400 Vrms and a duty cycle of about 5% to about 50% at about from slightly more than 0 Hz to about 10 Hz. More specific embodiments include radio frequency energy of not more than about 60 watts, a voltage from about 240 Vrms to about 300 Vrms and a duty cycle of 5% to 40% at 1 Hz, with possibly, the pulsed radio frequency energy being delivered for a maximum of 10 seconds. In one example, the generator can be set to provide pulsed radio frequency energy of not more than about 50 watts, a voltage of about 270 Vrms, and a duty cycle of about 10% at 1 Hz. Alternatively, the pulsed radio frequency energy could comprise radio frequency energy of not more than about 50 watts, a voltage of about 270 Vrms, and a duty cycle of about 30% at 1 Hz.

In still other embodiments of apparatus 102, different energy sources may be used, such as radiant (e.g. laser), ultrasound, thermal or other frequencies of electrical energy (e.g. microwave), with appropriate energy sources, coupling devices and delivery devices depending upon the desired clinical performance.

Additional details of the tissue penetration apparatus 102 are described in connection with FIG. 2. Referring to FIGS. 2 and 2A, the tubular body 104 comprises a cannula 206 such as a 0.050″ cannula having a central lumen 208 extending therethrough. The lumen 208 is dimensioned to slidably receive a guidewire 210 such as an 0.035″ guidewire. Of course, other dimensions of a guidewire may be utilized, including, for example and without limitation, a 0.032″ guidewire. In certain implementations, it may be desirable to electrically isolate the guidewire 210 from the cannula 206. This may be accomplished by providing a tubular insulation layer 212 positioned between the guidewire 210 and the cannula 206. In the illustrated embodiment, the insulation layer 212 comprises a coating or tubular sleeve surrounding the guidewire 210. A further tubular insulation layer 214 may be provided on the outside of the cannula 206 to electrically isolate the cannula 206 from the patient. The tubular insulation layer 214 may comprise a coating or tubular sleeve surrounding the cannula 206.

The coating may be included on any surface of any embodiment described herein. The coating may affect one or more properties of the applied surface. For example, the coating may affect one or more properties of smoothness, adhesion, flexibility, hardness, lubricity, and/or electrical resistance. It is contemplated that, in one embodiment, the coating on the guidewire 210 and the cannula 206 may comprise any of the same properties. However, in another embodiment, the coating on the guidewire 210 and the cannula 206 may comprise different properties. For example, in one embodiment, the insulation layer 214 on the cannula 206 may comprise the coating affecting any of the properties of electrical resistance, smoothness, and hardness. Further, the insulation layer 212 on the guidewire 210 may comprise the coating affecting any of the properties of smoothness, lubricity, and surface tension. Of course, in some embodiments, the coating may affect the properties of the insulation layers 212, 214 to different extents. For example, the coating may provide a greater degree of electrical resistance on cannula 206 than the electrical resistance provided on the guidewire 210.

The coating may be applied in multiple layers. In some instances, the device can be selectively masked prior to coating and/or the coating can be removed from selective areas after coating. The coating may be removed through any suitable method, such as grinding, abrasive blasting, laser ablation, etc. In some embodiments, the coating may terminate at a portion of the cannula 206 proximal to the distal end 202.

Any guidewire as described herein may comprise a coating to increase lubricity of at least a portion of the guidewire. The coating, in some instances, may not be for electrical resistance such that an interaction between the guidewire and an inner canula is primarily mechanical relative to clearance and friction.

Referring to FIG. 2B, a detailed view of the distal end 202 of cannula 206, having a guidewire 210 extending therethrough is illustrated. The guidewire 210 may comprise a pigtail or other curved distal end as is understood in the art.

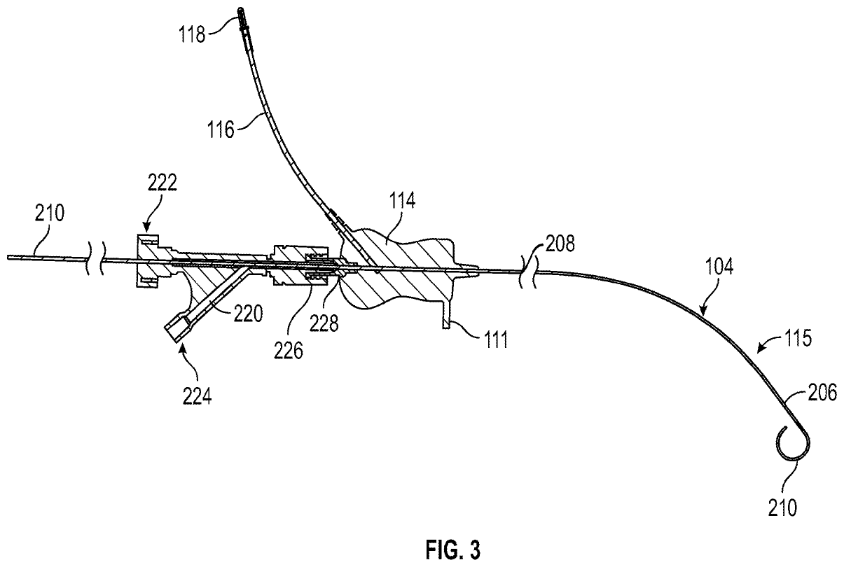

Referring to FIG. 3, the tissue penetrating apparatus 102 may additionally be provided with a Y connector 220, having a proximal guidewire access port 222 and a flush port 224 in communication with the distal region 106 such as via exit port 109 (FIG. 1) or cannula 206 central lumen 208. The Y connector 220 may be provided with a distal first connector 226 configured to cooperate with a second complementary connector 228 on the proximal end of the hub 114. First connector 226 and second connector 228 may be complementary components of a standard luer connector as is understood in the art. Alternatively, the hub 114 and Y connector 220 may be formed as an integral unit.

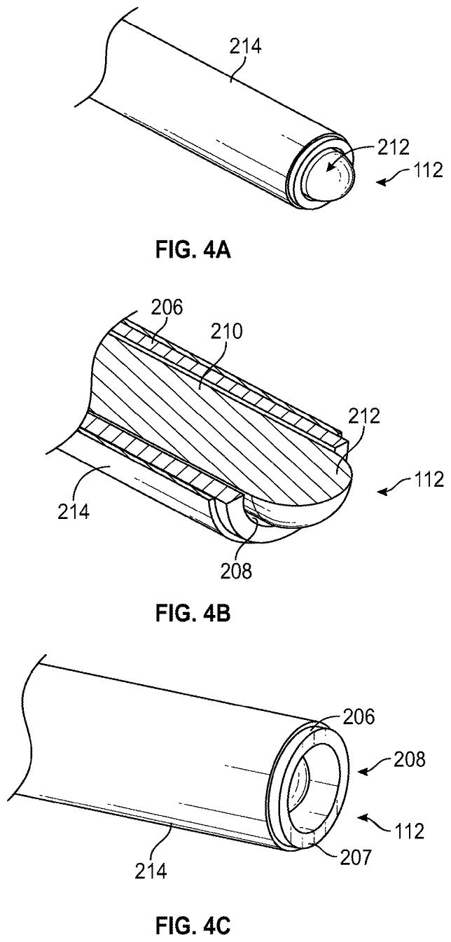

FIGS. 4A-4C illustrate the axial slidability of the guidewire 210 within the cannula 206. In FIG. 4C, the guidewire has been proximally retracted into the central lumen 208 so that the leading surface of the system is an annular charge transfer surface 207, which comprises a distal end face of the cannula 206. The insulation layer 214 on cannula 206 may extend distally all the way to the edge of the end face of the cannula 206, or to no more than 2 mm or 1 mm or less proximally of the end face of the cannula 206. The system thus permits delivering RF energy from either the guidewire alone, or the cannula alone or both, depending upon the desired clinical performance.

The separately insulated cannula 206 and guidewire can be configured to deliver bipolar electricity to the distal tip. The cannula 206 can be used as the ground path and replace the body pad or other electrode, which may provide desirable impedance characteristics depending upon the desired clinical performance.

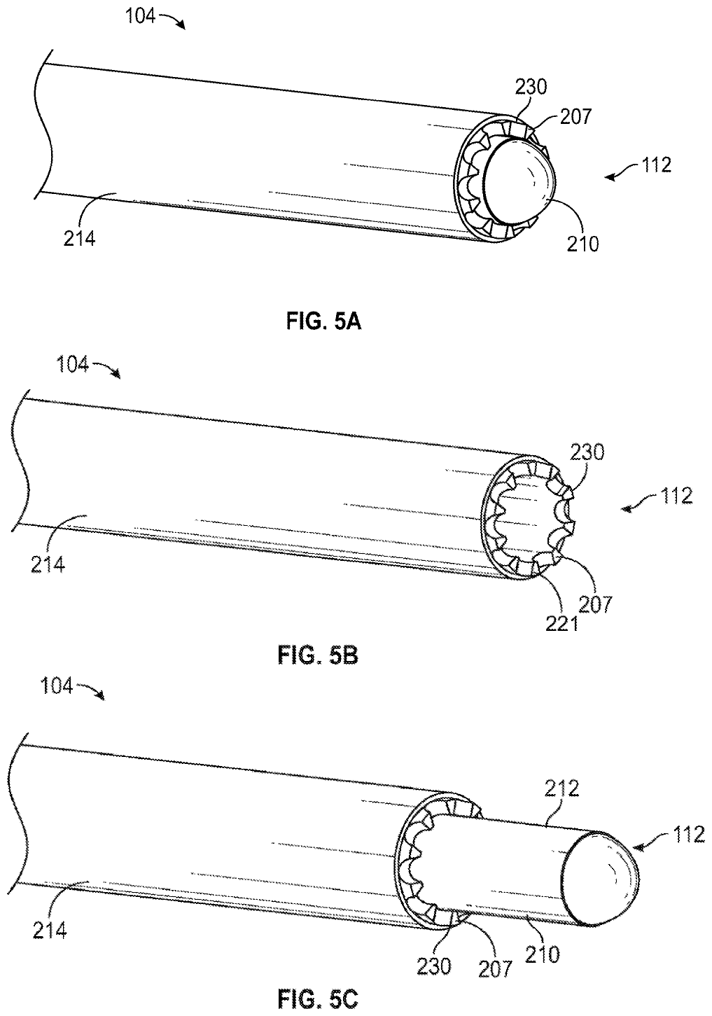

FIGS. 5A-5C illustrate a modified distal end face of the cannula 206. At least one distal extension 230 is carried by the cannula 206, to provide a site of enhanced energy density on the leading charge transfer surface 207 carried by the projection 221. At least two or four or more projections 221 may be provided, such as 10 as illustrated in FIG. 5B, creating scalloped surface with a plurality of circumferentially spaced apart distal transfer surfaces 207.

One method of delivering a large bore catheter in a single pass using the transseptal puncture system may be as follows.
1. Advance a guidewire (GW) into the superior vena cava (SVC) and deliver a large bore catheter (e.g., left atrial appendage occlusion device; mitral valve repair or replacement; intra atrial adjustable annuloplasty device) with a dilator to the SVC.
2. Withdraw GW inside of the dilator.
3. Withdraw the large bore sheath and the dilator down to the right atrium.
4. Steer the sheath and dilator into position in the interatrial septum, specifically tenting the septum with the dilator.
5. Deliver the cannula and GW into position with the cannula extending distally beyond the dilator and the GW distally beyond the cannula and in contact with the fossa ovalis.
5a. If necessary for positioning purposes, withdraw cannula proximal to the bend of the steerable sheath, then step 6.
6. Activate the distal tip of the GW with RF energy, and pass the GW through the septum and into the left atrium (LA).
7. Drive the cannula, dilator and sheath distally through the septum and into the LA.
8. If the cannula cannot pass through the septum into the LA, activate the distal tip of the cannula with RF energy and advance the cannula into the LA.
9. Drive the dilator and the large bore sheath over the access cannula and into the LA.
10. Withdraw the cannula and dilator, and introduce the index procedure catheter through the large bore sheath.

As will be appreciated by those of skill in the art, the GW and cannula can alternatively be simultaneously operated in monopolar mode; either the GW or cannula can be energized separately; or the GW and cannula can be operated in bipolar mode, depending upon the desired clinical performance.

Thus, referring to FIG. 6, there is illustrated a schematic cross-section of a portion of the heart 10. The right atrium 86 is in communication with the inferior vena cava 88 and the superior vena cava 90. The right atrium 86 is separated from the left atrium 16 by the intraatrial septum 18. The fossa ovalis 92 is located on the intraatrial septum 18. As seen in FIG. 6, a large bore transseptal sheath 12 may have a dilator 84, both riding over the cannula 206 and guidewire 210, all positioned within the right atrium 86.

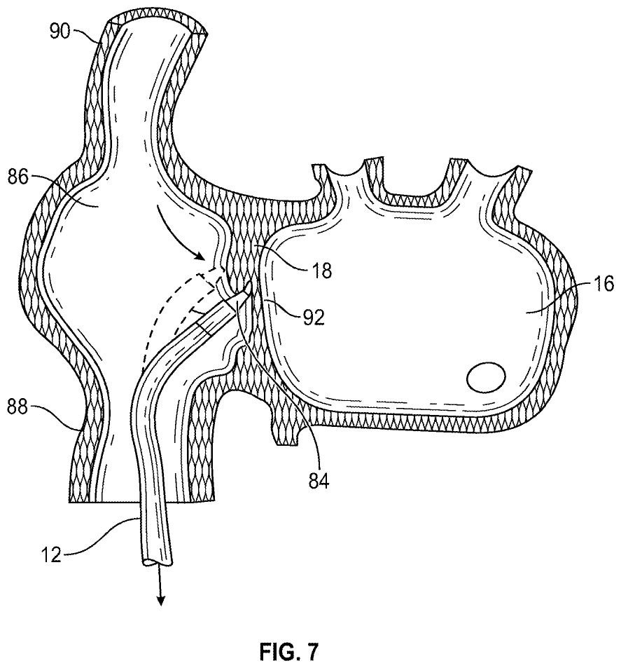

The combination of the sheath 12 with the dilator 84 having the transseptal cannula 206 and GW 210 extending distally therefrom, is then drawn proximally from the superior vena cava while a curved section of the sheath, alone or in combination with a preset curve at the distal region of dilator 84 and or cannula 206, causes the tip of the cannula—GW combination to “drag” along the wall of the right atrium 86 and the septum 18, by proximal traction until the tip pops onto the fossa ovalis 92, as shown in FIG. 7.

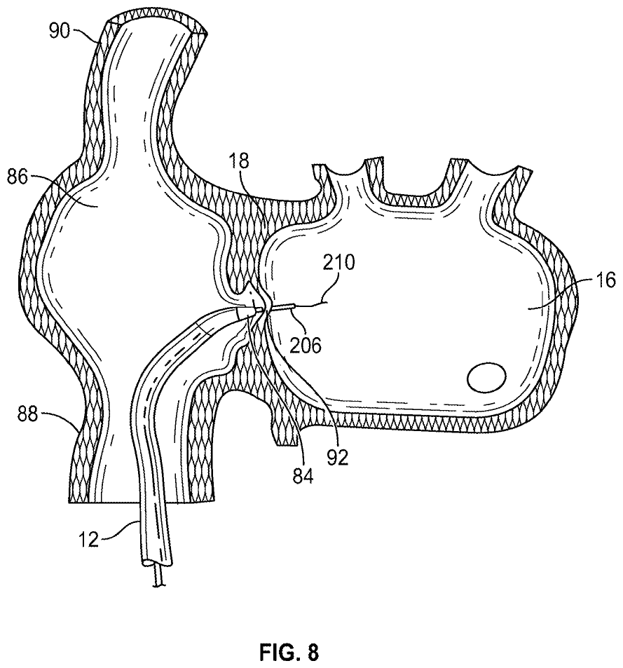

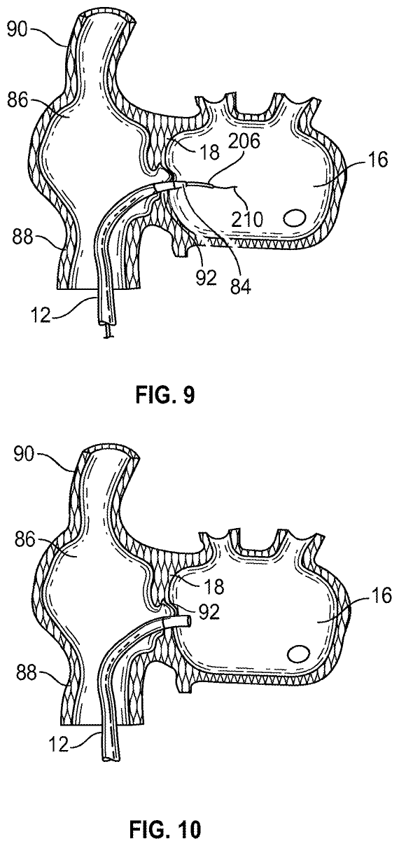

After the tip of the cannula—GW combination has been placed in the desired location against the fossa ovalis 92, RF energy is applied via the tip of the transseptal GW 210 to allow the GW 210 to pass through the septum into the LA. As previously described, RF energy may also be delivered via the distal end of the cannula 206 if desired. See FIGS. 8 and 9.

One medical technique is to confirm the presence of the tip of the transseptal GW 210 within the left atrium 16. Confirmation of such location of the tip of the transseptal GW 210 may be accomplished by monitoring the pressure sensed through a transseptal GW lumen or an annular lumen defined between the GW 210 and the inside surface of the cannula 206 central lumen to ensure that the measured pressure is within the expected range and has a waveform configuration typical of left atrial pressure. Alternatively, proper position within the left atrium 16 may be confirmed by analysis of oxygen saturation level of the blood drawn through an available lumen; i.e., aspirating fully oxygenated blood. Finally, visualization through fluoroscopy alone, or in combination with the use of dye, may also serve to confirm the presence of the tip of the transseptal cannula 206 and GW 210 in the left atrium 16.

After placing the transseptal cannula tip within the left atrium 16, the tip of the dilator 84 is advanced through the septum 18 and into the left atrium 16, as shown in FIG. 9. When the tapered tip of dilator 84 appears to have entered the left atrium 16, the transseptal cannula 206 may be withdrawn. The large bore sheath 12 may then be advanced into the left atrium 16, either by advancing the sheath 12 alone over the dilator 84 or by advancing the sheath 12 and dilator 84 in combination. The dilator 84 may then be withdrawn from sheath 12 when the latter has been advanced into the left atrium, thus leaving the main lumen of sheath 12 as a clear pathway to advancing further large bore diagnostic or therapeutic instruments into the left atrium.

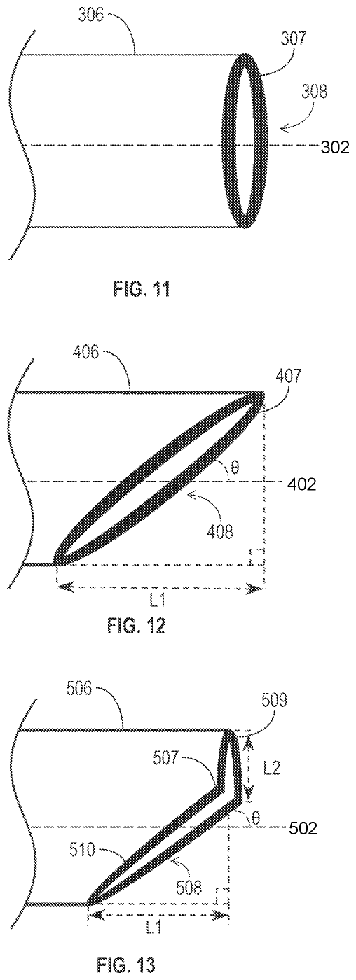

FIGS. 11-15 are views of various examples of a modified distal end face of a cannula, according to some embodiments. For example, FIG. 11 illustrates a cannula 306 comprising a distal end face 307 being generally perpendicular to a longitudinal axis 302 of the cannula 306. By way of another example, FIG. 12 illustrates a cannula 406 comprising a distal end face 407 being generally non-orthogonal relative to a longitudinal axis 402 of the cannula 406.

By way of further example, FIG. 13 illustrates a cannula 506 comprising a distal end face 507 having a first section 509 oriented at a first angle with respect to a longitudinal axis 502 of the cannula 506 and a second section 510 oriented at a second, different angle relative to the longitudinal axis 502 of the cannula 506. Each or either of the first section 509 and second section 510 may reside on a plane such that each section of the end face is substantially linear when viewed from a side elevational view. Alternatively, one or both of the bevel end faces may be nonlinear, in which case the angles discussed herein will be defined with respect to a transverse plane which intersects the two endpoints of a subject first or second section.

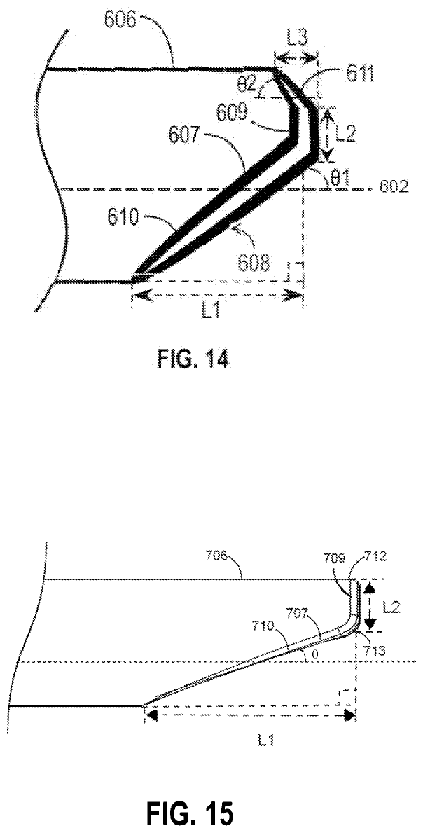

By way of even further example, FIG. 14 illustrates a cannula 606 being generally similar to cannula 506 and further comprising a blunt configuration. The cannula 606 can comprise a distal end face 607 having a first section 609 oriented at a first angle with respect to a longitudinal axis 602 of the cannula 606, a second section 610 oriented at a second, different angle relative to the longitudinal axis 602 of the cannula 606, and a third section 611 oriented at a third, different angel relative to the longitudinal axis 602 of the cannula. At least one of the first section 609, second section 610, and third section 611 may reside on a plane such that each section of the end face is substantially linear when viewed from a side elevational view. Alternatively, at least one of the end faces may be nonlinear, in which case the angles discussed herein will be defined with respect to a transverse plane which intersects the two endpoints of a subject first or second section. The third section 611 may function as a radially outwardly facing ramped deflection surface, as described herein.

By way of further example, FIG. 15 illustrates a cannula 706 comprising a distal end face 707 having a first section 709 oriented at a first angle with respect to a longitudinal axis of the cannula 506 and a second section 710 oriented at a second, different angle relative to the longitudinal axis of the cannula 706. The cannula 706 may comprise one or more rounded edges 712, 713 along an intersection between one or more sections of the cannula 706.

Unless otherwise noted, reference numerals in FIGS. 11-15 refer to components that are the same as or generally similar to the corresponding components in the remaining figures discussed herein (e.g., the reference numerals may refer to components with the same or similar last two digits as provided in the remaining figures). It will be understood that the features described with reference to any one of cannulas 306, 406, 506, 606, 706 as shown in FIGS. 11-15, respectively, can be used with any of the other cannulas 306, 046, 506, 606, 706 or any other embodiment described and/or contemplated herein.

FIG. 11 illustrates an embodiment of a cannula 306 comprising a distal end face 307 residing on a transverse plane that resides generally perpendicular to a longitudinal axis 302 of the cannula 306.

FIG. 12 illustrates an embodiment of a cannula 406 comprising a substantially planar distal end face 407 that is oriented at a non-orthogonal angle θ relative to a longitudinal axis 402 of the cannula 406. The non-orthogonal orientation may in some instances, provide for increased tissue penetrating properties. The non-orthogonal or bevel orientation may advantageously minimize any unwanted coring of the tissue and, consequently, may increase a tissue thickness capable of being readily penetrated by the cannula 406. For example, the bevel tip may be able to facilitate penetration of tissue at least equal to length L1 of the angled distal end face 407 in a longitudinal direction. The angled distal end face 407 may advantageously penetrate the tissue in a mechanical and/or electrical manner.

FIG. 13 illustrates an embodiment of a cannula 506 comprising a distal end face 507 having a first section 509 that is positioned generally perpendicular to a longitudinal axis 502 of the cannula 506 and a second section 510 that is positioned at a non-orthogonal angle θ relative to the longitudinal axis 502 of the cannula 506. The combination of the first section 509 and the second section 510 may in some instance, improve the ability of the cannula 506 to create a tissue penetration with the leading first end face and facilitate tissue penetration with the inclined, second section 510. This compound functionality tip enables penetration with the first, penetrating section 509 followed by dilatation of the penetration in response to distal advance of the inclined second section 510 through the penetration caused by penetration section 509. This configuration may minimize the risk of coring by creating a small tissue flap resulting in an expandable tissue aperture for receiving devices therethrough.

The first section 509, in some embodiments, may include an arcuate end face extending between a first inflection point and a second inflection point. An arc length of the arcuate end face may be generally less than about 180 degrees and greater than about 45 degrees.

The length L2 of the first section 509 (as measured along a direction perpendicular to a longitudinal direction) may be optimized due to the circumstances of use of the cannula 506. The surface area the first section 509 is related to the length L2 and affects the amount of force that is applied to a targeted tissue during treatment. The force is applied through manipulation of a handle of the system that is manipulated by a user. The force applied by the first section 509 onto the targeted tissue contributes to the treatment by applying a mechanical component to the coring solution. For example, when appropriate force is applied, splits or tears are formed in the targeted tissue that substantially reduces or prevents coring. The length L2 may be optimized considering these benefits.

As length L2 increases, length L1 may decrease depending on any adjustment to the angle θ. Length L2 may be about 5% to about 75% of a diameter of the distal end face 507 and/or of a distal section of the cannula 506. In some instances, length L2 may be about 10% to 50% or, more specifically, about 25% to about 30% of the diameter of the distal end face 507 and/or of a distal section of the cannula 506. In one embodiment, the length L1 may be at about 20% to about 50% of the diameter of the distal end face 507 and/or of the distal section of the cannula 506. In some embodiment, the length L1 may be from about 25% to about 30% of the diameter of the distal end face 507 and/or of the distal section of the cannula 506.

Length L1 may be about 1 mm to about 2.5 mm. For example, length L1 may be about 1.5 mm to about 2 mm or, in some instances, about 1.75 mm. It is contemplated that if length L1 is too large, the inclined second section 510 (that, in some embodiments, may not comprise a coating) may be left open to a blood pool and may in some instances, cause a puncture ability to become inconsistent. Further, if length L1 is too short, the inclined second section 510 may not provide any additional benefit as described herein.

Length L2 may be about 0.25 mm to about 0.75 mm. For example, length L2 may be about 0.4 mm to about 0.6 mm or, in some instances, about 0.5 mm. It is contemplated that if length L2 is too short relative to length L1, the first section 509 may become too sharp and reduce the efficacy of the first section 509 during use.

The angle θ may be altered, in some configurations, to provide for a desired length L2 to maintain non coring tissue penetrating properties of the first section 509, while also providing for a desired length L1 to achieve the tissue dilatation properties of the second section 510. For example, length L1 may be determined based on a target tissue thickness such that length L1 is formed to be equal to or greater than the target tissue thickness. For example, as angle θ approaches 0°, length L1 will increase and, thereby, increasing a tissue thickness that the cannula 506 may penetrate while reducing the risk of coring.

In one embodiment, angle θ may be about 10° to about 70°. In some instances, angle θ may be about 15° to 50° or, more specifically, about 20° to 30°. In another embodiment, angle θ may be about 15° to about 35°. For example, Angle θ may be about 20° to about 30° or, in some instances, about 25° or about 26°. In some embodiments, angle θ may be about 40° to about 50°.

FIG. 14 illustrates an embodiment of a cannula 606 comprising a blunt configuration which may be in the form of a radially outwardly facing ramped deflection surface, for example the third section 611. The deflection surface, for example the third section 611, in some instances, may be a radiused curve or linear surface which inclines radially inwardly in the distal direction. Deflection surface, for example the third section 611, may advantageously allow the needle to advance distally through the dilator while minimizing the risk of disrupting particles from the inner diameter of the dilator with the relatively sharp leading edge of the first section 609.

Length L1 and angle θ₁ may comprise any of the same dimensions as discussed in reference to FIG. 14. Length L3, length L2, and angle θ₂ may be any dimensions that a person of ordinary skill in the art may desire.

FIG. 15 illustrates an embodiment of a cannula 706 that may be the same or generally similar to cannula 506 as described in connection with FIG. 13, except as otherwise described herein. Cannula 706 may comprise a distal end face 707 having a first section 709 that is positioned generally perpendicular to a longitudinal axis of the cannula 706 and a second section 710 that is positioned at a non-orthogonal angle relative to the longitudinal axis of the cannula 706. The cannula 706 may comprise one or more rounded edges each located along an intersection between one or more sections of the cannula 706. The one or more rounded edges may provide various advantages, such as minimizing risks associated with a sharp edge (e.g., skiving while advancing the cannula).

The cannula 706 may comprise a rounded edge 712 located at an intersection between the first section 709 and a sidewall of the cannula 706. The rounded edge 712 may extend through an entire thickness of the cannula 706 sidewall. It is contemplated that the rounded edge 712 may produce a third section of the distal end face 707 that may be less pronounced than the deflection surface 611 of the cannula 606 In some instances, the rounded edged 712 may only extend partially through a thickness of the sidewall of the cannula 706.

Length L1, length L2, and angle θ may be any dimensions that a person of ordinary skill in the art may recognize or desire. In one embodiment, any of the length L1, length L2, and angle θ may comprise any of the same dimensions as discussed with reference to FIG. 14. In an embodiment, length L1 and length L2 may comprise dimensions such that angle θ may be between 15° and 50°. In one such embodiment, length L1 and length L2 may comprise dimensions such that angle may be between 20° and 30°. In another such embodiment, length L1 may be about 1.75 mm, length L2 may be about 0.50 mm and angle θ may be between about 25° and 26°, for example 25.37°. It is contemplated that the angle may be configured in any manner to reduce coring when puncturing the tissue.

Length L2 may contact the desired region of tissue, for example, the septum and supply energy to the septum and create a weakness in the septum to provide entry into the left atrium. It is contemplated that the weakness may permit the creation of the puncture, either directly from the delivery of the energy or through the application of force. Length L1 being angled away from length L2 may permit a gradual increase in puncture size. This gradual increase is contemplated to reduce the occurrence of coring when advancing the cannula 706 into the left atrium.

A radius of curvature of the rounded edge 712 may be altered, in some configurations, to maintain the beneficial properties of the rounded edge 712, while also minimizing any reduction in length of the first section 709. The radius of curvature of the rounded edge 712 may be about 0.05 mm to about 0.25 mm. For example, the radius of curvature of rounded edge 712 may be about 0.1 mm to about 0.2 mm or, in some instances, about 0.13 mm or about 0.15 mm.

The cannula 706 may comprise a rounded edge 713 located at an intersection between the first section 709 and the second section 710. A radius of curvature of the rounded edge 713 may be altered, in some configurations, to maintain the beneficial properties of the rounded edge 713, while also minimizing any reduction in length of the first section 709 and/or the second section 710. The radius of curvature of the rounded edge 713 may be about 0.05 mm to about 0.25 mm. For example, the radius of curvature of rounded edge 713 may be about 0.1 mm to about 0.2 mm or, in some instances, about 0.13 mm or about 0.15 mm.

One or more materials may be selected and/or applied to at least a portion of a distal end face of a cannula to alter various properties of cannula. While the following discussion specifically references the embodiment of FIG. 15, it will be understood that any of the features described in connection with altering various properties of the cannula can be used with any of the embodiments described and/or contemplated herein (such as cannula 306, 406, 506, 606).

The thermal and/or electrical conductivity of the cannula 706, or in some instances of the distal end face 707, may be altered through a selection of a particular base material and/or through application of a coating to the distal end face 707. In some embodiments, a material (e.g., gold) that is more thermally and/or electrically conductive and/or has a different density than the material of the conductive portion of the body of the cannula 706 may be applied as a coating to at least a portion of the distal end face 707. The increased conductivity of the coating material may advantageously enhance the application of thermal and/or electrical treatment to the target tissue. In some instances, the coating material may decrease a power output and/or a push force required to penetrate a target tissue during use. For example, a material may be selected that is about 25% to about 50% more thermally and/or electrically conductive than the cannula body material, which may be stainless steel. In some instances, a material having a greater density (e.g., silver) than a remaining portion of the body of the cannula 706 may be selected to increase a radiopaque quality of the distal end face 707. The increased radiopacity may advantageously enhance visualization of the distal end face 707 relative to the remaining body of the cannula 706 during a treatment procedure. It will also be understood that at least a portion of the distal end face 707 may be formed of the more conductive and/or radiopaque material relative to a cannula body material instead of solely applying a coating to the distal end face 707.

In some instances, the material properties (e.g., thermal and/or electrical conductivity) may be altered as between the first section 709 and the second section 710 of the distal end face 707 to further increase the tissue penetration capabilities of the cannula 706. For example, the first section 709 may comprise a first material, and the second section 710 may comprise a second material. The first material may have a thermal and/or electrical conductivity that is greater than or lesser than a thermal and/or electrical conductivity of the second material. The first section 709, in some embodiments, may comprise a first coating, and/or the second section 710 may comprise a second coating. In some instances, the first section 709 may comprise a first coating, but the second section 710 is uncoated. The first coating may have a thermal and/or electrical conductivity that is greater than or lesser than a thermal and/or electrical conductivity of an exposed surface (e.g., coated or uncoated) of the second section 710. In some examples, only one of the first section 709 or the second section 710 comprises a coating having increased thermal and/or electrical conductive properties.

The tubular cannula can be formed of any material that a person of ordinary skill in the art may desire. In one implementation, the tubular cannula comprises stainless-steel. The end face of the inclined, tissue dilating second section 710 comprises an exposed inclined end face of the stainless-steel cannula. In some embodiments, as described herein, an arcuate end face of the first section 709 may be provided with an increased conductivity coating such as gold. Of course, any material or combination of materials may be utilized and the aforementioned is provided as a non-limiting example. The material or combination of materials may have properties related to any of conduciveness, high material strength, corrosion resistance, radiopacity, sterilization compatibility, or combinations thereof.

The coating may in some embodiment, be configured as an insulation layer on the cannula 706. As previously discussed, the coating may affect one or more properties of the cannula 706, including, for example electrical resistance. In some embodiments, the coating may be applied along the cannula 706 except at the distal end face of the cannula 706 to define an electrode tip. In one embodiment, the thickness of the coating may be gradually reduced near the distal end of the cannula 706 to blend into the distal face. In one embodiment, a distal edge of the coating may comprise a fillet to reduce any sharp edges at the distal end face of the cannula 706. In an embodiment, the distal end face of the cannula 706 and the coating may be filleted to reduce sharp edges and may thus, reduce coring and other undesired trauma at the septum. However, in another embodiment (not shown), the coating and/or the distal end face of the cannula may be chamfered or otherwise blended.

The edge of the coating may be blended according to a method comprising of grinding, laser processing, electropolishing, additive manufacturing, electrical discharge machining, machining, or any other method that may be desired.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially planar” is recited with respect to a feature, it is understood that in further embodiments, the feature can have a precisely planar orientation.

Any reference throughout this specification to “certain embodiments” or the like means that a particular feature, structure or characteristic described in connection with that embodiment is included in at least one embodiment. Thus, the quoted phrases, or variations thereof, as recited throughout this specification are not necessarily all referring to the same embodiment or embodiments.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

What is claimed is:

1. A transseptal crossing system comprising:
a sheath comprising an elongate tubular sheath body having a sheath lumen extending through the sheath body; and
an elongate tubular body comprising:
an electrically conductive sidewall defining a central lumen, the central lumen being configured to receive a guidewire and to permit at least a portion of the guidewire to extend through a distal opening of the electrically conductive sidewall; and
a distal end section comprising a distal end surface being in electrical communication with the electrically conductive sidewall, the distal end surface comprising:
a first section being positioned generally perpendicular to a longitudinal axis of the elongate tubular body; and
a second section being positioned at a non-orthogonal angle relative to the longitudinal axis of the elongate tubular body,
wherein at least one of the first section or the second section is configured to deliver energy to a target tissue.

2. The transseptal crossing system of claim 1, wherein the first section of the distal end surface comprises a length of at least about 20% of a diameter of the distal end section of the elongate tubular body.

3. The transseptal crossing system of claim 1, wherein a length of the first section is at most about 50% of a diameter of the distal end section of the elongate tubular body.

4. The transseptal crossing system of claim 1, wherein a length of the first section is between about 25% and about 30% of a diameter of the distal end section of the elongate tubular body.

5. The transseptal crossing system of claim 1, wherein the non-orthogonal angle of the second section is at least about 30°.

6. The transseptal crossing system of claim 1, wherein the non-orthogonal angle of the second section is at most about 70°.

7. The transseptal crossing system of claim 1, wherein the non-orthogonal angle of the second section is between about 40° and about 50°.

8. The transseptal crossing system of claim 1, wherein the first section comprises a first material, wherein the second section comprises a second material, and wherein the first material has a first conductive property different than a second conductive property of the second material.

9. The transseptal crossing system of claim 8, wherein the first material comprises a coating on the first section.

10. The transseptal crossing system of claim 8, wherein the second material comprises a coating on the second section.

11. The transseptal crossing system of claim 1 further comprising a dilator being configured to be positioned through the sheath lumen, the dilator having a dilator lumen extending through a dilator body and being configured to receive the elongate tubular body.

12. The transseptal crossing system of claim 1 further comprising a tubular insulation layer surrounding the electrically conductive sidewall and leaving the distal end section exposed.

13. A transseptal crossing system comprising:
a sheath comprising an elongate tubular sheath body having a sheath lumen extending through the sheath body; and
an elongate tubular body comprising:
an electrically conductive sidewall defining a central lumen, the central lumen being configured to receive a guidewire and to permit at least a portion of the guidewire to extend through a distal opening of the electrically conductive sidewall; and
a distal end section comprising a distal end surface being in electrical communication with the electrically conductive sidewall, the distal end surface comprising:
a first material comprising a first conductive property; and
a second material comprising a second conductive property, the second conductive property being different than the first conductive property,
wherein at least a portion of the distal end section is configured to deliver energy to a target tissue.

14. The transseptal crossing system of claim 13, wherein the first material comprises a coating on a portion of the distal end surface.

15. The transseptal crossing system of claim 13, wherein the first material comprises gold.

16. The transseptal crossing system of claim 13 further comprising a dilator being configured to be positioned through the sheath lumen, the dilator having a dilator lumen extending through a dilator body and being configured to receive the elongate tubular body.

17. The transseptal crossing system of claim 13 further comprising a tubular insulation layer surrounding the electrically conductive sidewall and leaving the distal end section exposed.
